(19) United States
(12) Patent Application Publication
(10) Pub. No.: US 2019/0382818 A1
(43) Pub. Date: Dec. 19, 2019

(54) METHOD FOR PREPARATIVE PRODUCTION OF LONG NUCLEIC ACIDS BY PCR

(71) Applicants: Helmut Merk, Berlin (DE); Volker Erdmann, Berlin (DE); Wolfgang Stiege, Berlin (DE)

(72) Inventors: Helmut Merk, Berlin (DE); Volker Erdmann, Berlin (DE); Wolfgang Stiege, Berlin (DE)

(21) Appl. No.: 16/251,184

(22) Filed: Jan. 18, 2019

Related U.S. Application Data
(63) Continuation of application No. 15/388,164, filed on Dec. 22, 2016, now abandoned.
Application No. 15/388,164 is a continuation of application No. 14/605,513, filed on Jan. 26, 2015, now abandoned.
Application No. 14/605,513 is a continuation of application No. 13/630,643, filed on Sep. 28, 2012, now abandoned.
Application No. 13/630,643 is a continuation of application No. 12/365,702, filed on Feb. 4, 2009, now abandoned.
Application No. 12/365,702 is a continuation of application No. 10/472,003, filed on Sep. 8, 2005, now abandoned.

(30) Foreign Application Priority Data
Mar. 16, 2001 (DE) 101 13 265.4

(51) Int. Cl. C12P 19/34; C07K 14/47; C12N 15/68; C12N 15/66; C12N 15/10

(52) U.S. Cl. CPC C12P 19/34; C07K 14/4703; C12N 15/10; C12N 15/66; C12N 15/68

(57) ABSTRACT

The invention relates to a method for preparative production of long nucleic acids by PCR. The method involves the following hybridization steps: a) a nucleic acid base sequence is hybridized on the 3′ and 5′ ends with an adapter primer; b) the product from step a) is hybridized on the 3′ and 5′ ends with an extension primer containing an extension sequence, wherein a nucleic acid with extension sequences amplified and enlarged in the 3′ and 5′ ends of the nucleic acid base sequence is then formed from the nucleic acid base sequence. The invention also relates to different applications of the inventive method.

### CROSS REFERENCE TO RELATED APPLICATION

This application is a continuation of U.S. application Ser. No. 15/388,164, filed Dec. 22, 2016, which is a continuation of U.S. application Ser. No. 14/605,513, filed Jan. 26, 2015, now abandoned, which is a continuation of U.S. application Ser. No. 13/630,643, filed: Sep. 28, 2012, now abandoned, which is a continuation of 12/365,702, filed Feb. 4, 2009, now abandoned, which is a continuation of U.S. Ser. No. 10/472,003, filed Sep. 8, 2005, now abandoned, which is a National Stage Entry of PCT/DE02/01047, filed Mar. 18, 2002, now expired, which claims the benefit of German Application No. 101 13 265.4, filed Mar. 16, 2001. All of the above applications are incorporated herein by reference.

### FIELD OF THE INVENTION

The invention relates to a method for preparative production of long nucleic acids by PCR and to different applications for such a method. As a preparative scale are considered the obtained amounts of nucleic acids which are suitable for an immediate application in cell-free protein biosynthesis systems and/or in vitro transcription systems. Long nucleic acids are such nucleic acids which contain, in addition to a nucleic acid base sequence (arbitrary length) coding for a protein, further sequences, in particular regulatory sequences of more than 50, even more 70 nucleotides each. Nucleic acids may be DNA or RNA, but also PNA.

### BACKGROUND OF THE INVENTION

Proteins for biotechnological and medical applications are needed with a high purity, in particular however also with a high amount, i.e. in the mg or g range. In the case of larger proteins, a classical synthesis is nearly impossible and in any case uneconomic.

One possibility to produce proteins on a larger scale is genetic production. For this purpose, cloned DNA coding for the desired protein is introduced as a foreign DNA in the form of vectors or plasmids, in particular prokaryotic cells. These cells are then cultivated, whereby the proteins coded for by the foreign DNA are expressed and obtained. In this way considerable amounts of protein may be obtained, however, the insofar known methods, in particular cloning, are expensive. Furthermore, the cells are in most cases only transiently transfected, and in exceptional cases only stably immortalized. A continuous production of protein therefore requires a permanent supply of fresh cells, which in turn have to be produced by means of the above expensive measures.

Another approach is the so-called cell-free in vitro protein biosynthesis. Herein, biologically active cell extracts are used, which are to a high extent freed from naturally occurring cellular nucleic acids and which are reacted with amino acids, energy supplying substances and at least one nucleic acid. The added nucleic acid codes for the protein to be produced. If DNA is used as the nucleic acid, the presence of a DNA dependent RNA polymerase is required. Of course, RNA, mRNA may also directly be used. In this way, not only such proteins which may also be produced genetically can be produced in a short time and at comparably moderate expenses, rather such proteins can even be produced which are for instance cell toxic and consequently cannot be expressed at all by the usual genetic cell systems at a notable degree. However, it is necessary to produce the added nucleic acid itself, which again is expensive by means of genetic methods. In addition, it is often desirable to introduce regulatory sequences not naturally linked with a protein sequence and other sequences, such as spacers, in order to improve the efficiency of the protein synthesis.

An alternative to the genetic production of complete nucleic acids to be used in the cell-free protein biosynthesis is the so-called expression PCR. In these connections, the efficient introduction of regulatory sequences (and of other sequences promoting the translation efficiency) into a nucleic acid to be produced plays a special role for the amplification. For the introduction of such further sequences into a target nucleic acid, very long PCR primers are necessary. Long primers are on the one hand expensive to produce and increase on the other hand the probability of the generation of inhomogeneous PCR products.

### PRIOR ART

From the document USA-Pat. No. 5,571,690 it is known in the art to produce a nucleic acid in a preparative scale by means of PCR, wherein the nucleic acid to be amplified already contains all necessary regulatory sequences. The insofar known measures do therefore not permit an introduction of other, better regulatory sequences or a replacement of existing regulatory sequences by such other better sequences. Further, the nucleic acid obtained from the amplification cannot immediately be used in the protein synthesis. Finally, a specific nucleic acid from a nucleic acid mixture cannot be amplified, with a simultaneous conversion of the target gene for the protein biosynthesis coded by the specific nucleic acid.

From the document WO-A-9207949 it is known in the art to produce and amplify in several steps a nucleic acid with a sequence coding for a protein and with a regulatory sequence. In a first step, the sequence coding for the protein is amplified with a standard PCR. An upstream hybridizing primer serves for the introduction of an adapter sequence for a so-called “overlap extension PCR”. In a parallel second step the hybridization partner for the overlap extension PCR is prepared. For this purpose, two partially complementary primers are hybridized and filled up. The obtained product carries at the 3′ end the sequence of the adapter sequence being homologous to the 5′ end of the amplicon of the first step as well as a promoter sequence and regulation elements for the cell-free protein biosynthesis. The third step finally is the overlap and extension reaction. The products of the first two steps are hybridized, filled up so to form a double strand and finally amplified with further primers. In this final amplification, an additional sequence is incorporated via a primer ahead of the promoter, with the purpose of an improved transcription. In two further steps, the transcription as well as the translation in a cell-free system takes place.

It is disadvantageous, herein, that in total four steps are necessary for obtaining the desired mRNA. Further, the 3′ sequences being necessary for the protein biosynthesis in prokaryotic systems are lacking. Further, no affinity tag sequences or the like are introduced, by means of such sequences the purification of the obtained protein being facilitated. Due to the complexity of the method and the lack of 3′ sequences for prokaryotic systems, the insofar known method should neither be useful for prokaryotic systems nor for an application to nucleic acid mixtures (cDNA or genome libraries).

In the document Martemyanov, K. A., et al.; FEBS Lett. 414:268-270 (1997), a method is described which is similar to that of the document WO-A-9207949. There are differences in that a sequence homology between the 5′ end of the up-stream and the 3′ end of the downstream primer exists. Thereby a multimerization takes place, and at long last a polyprotein is produced which is then again cleft into monomers. In addition it is disadvantageous, in this variant, that polyproteins of very few proteins only can chemically be cleft. Furthermore, the yield is relatively low.

From the document Nakano, H., et al.; Bio-technol. Bioeng. 64:194-199 (1999), a special protein bioreactor for the expression of PCR products in Escherichia coli lysate is known in the art. Therein, standard PCR products are used without special methods for the generation thereof. The PCR product is expressed in a distinctly poorer condition than in a plasmid.

All above documents relate to eukaryotic systems.

### BRIEF DESCRIPTION OF THE DRAWINGS

FIG. 1 shows the employed primer sequences.

FIG. 2 shows a diagrammatic representation of a one-step PCR according to the invention with four primers.

FIG. 3 shows that all sequence regions except 0 (lower curve) cause an increase in the protein synthesis.

FIG. 4 shows that by the phage T7 gene 10 transcription terminator the synthesis can be at least improved by a factor 2.8.

FIG. 5 shows that the spacer sequence causes an approximate 2-fold increase in the expression.

FIG. 6 shows that the half-life period of the PCR product is approximately 100 minutes, which corresponds to the time when the H-FABP synthesis reaches a horizontal line.

FIG. 7 shows the structure and properties of a biotin-marked primer.

FIG. 8 shows the influence of streptavidin on protein synthesis.

### TECHNICAL OBJECT OF THE INVENTION

The invention is based on the technical object to provide a method for the production of long nucleic acids, in particular with protein sequences as well as with selected regulatory sequences, wherein said method needs low expenditure, generates high amounts of product nucleic acids, is suited without additional expenditure for prokaryotic systems, and by means of which an amplification of defined nucleic acids from nucleic acid libraries is possible.

### Basics of the Invention

For achieving the above technical object, the invention teaches a method for preparative production of long nucleic acids by means of PCR and involving the following hybridization steps: a) a nucleic acid base sequence is hybridized on the 3′ and 5′ ends with an adapter primer, b) the product from step a) is hybridized on the 3′ and 5′ ends with an extension primer containing an extension sequence, wherein a nucleic acid enlarged by the extension sequences and amplified on the 3′ and 5′ ends of the nucleic acid base sequence is then formed from the nucleic acid base sequence.

A nucleic acid base sequence is a sequence coding for a protein. In particular this may be a gene, however also sequences from intronless genomes. The extension sequences may in particular be sequences comprising a regulatory sequence and/or sequences comprising a ribosomal binding sequence. The adapter primers are comparatively short. One part of an adapter sequence is specific for the nucleic acid base sequence, another part is constant and hybridizes an extension sequence.

This means that it is not necessary to use respectively “fitting” long extension sequences for different nucleic acid base sequences. Rather, the comparatively short adapter primers only have to be adjusted to a defined nucleic acid base sequence, whereas the extension sequences may so to speak be universal, i.e. for different nucleic acid base sequences can always be used the same or several few selected extension sequences. Thus the extension sequences produced in an expensive way can be put to a wider use, and for a specific nucleic acid base sequence only the adapter sequences need to be produced. This is however little expensive, since the adapter sequences can be relatively short.

This permits for instance to add a regulatory sequence as well as a ribosomal binding sequence, each via one of the extension primers, to a nucleic acid base sequence, and that even in one PCR step. Thus a nucleic acid can be obtained which results in a particularly high transcription and/or translation efficiency in a prokaryotic system of the cell-free protein biosynthesis.

A particular advantage of the method according to the invention is that it is a generally applicable method for arbitrary coding sequences.

The hybridization with a primer on the 3′ and 5′ ends relates in particular to double-stranded nucleic acids, the hybridization of the primer respectively occurring on the 5′ end of the sense and antisense strands. Referred to the single strand, the hybridization with the various primers described above and below takes place on the respective 5′ end.

### Embodiments of the Invention

Of independent importance is a embodiment of the invention, wherein the product from step b) can be hybridized in a step c) on the 3′ and 5′ ends with respectively one amplification primer, and an amplified nucleic acid end sequence is formed. The amplification primers, too, are on the one hand relatively short and universally applicable, and consequently easily available. By means of the amplification primers, moreover further (shorter) sequences can be added on the ends, such sequences further increasing the translation efficiency. By means of the short amplification primers, variations and modifications on the ends of the nucleic acids can also easily be introduced. This is in particular advantageous, since thus for variations and modifications no different extension primers need to be produced, which again would be expensive in a disturbing manner.

An example for a variation or modification is the incorporation of a biotin residue, coupled on the 5′ end of the amplification primer. Hereby is obtained, after incubation of the nucleic acid end sequence with for instance biotin-binding streptavidin, a nucleic acid end sequence being stable against exonuclease disintegration which shows an increase in the half-life period in an in vitro protein biosynthesis system compared to a not stabilized nucleic acid end sequence by a multiple, typically more than 5 times, for instance from approx. 15 min to approx. 2 h. There are obtained stabilities which are comparable to those of circular plasmids and thus can replace them in an equivalent manner. An alternative is a stabilization by means of digoxigenin binding anti-digoxigenin antibodies. The stabilizing group may be provided on both ends of the nucleic acid end sequence. Another example is a modification with an affinity tag or a sequence coding therefore or an anchor group or a sequence coding therefor. An anchor group permits an immobilization by binding the anchor group to a solid body surface with matched binding sites. The anchor group may be disposed at the nucleic acid itself, however a sequence coding therefore may also be provided.

The adapter primers typically contain <70, in particular 20 to 60 nucleotides. The extension primers typically contain ≥70, even 90 and more nucleotides. The amplification primers finally typically contain <70, in most cases <30 nucleotides, typically >9 nucleotides. Just the adapter primers need to be specifically adjusted to a defined nucleic acid base sequence, which requires little efforts because of the relatively short sequences.

Advantageously, the steps a), b) and as an option the step c) are performed in a PCR solution containing the nucleic acid base sequence, the adapter primers, the extension primers and as an option the amplification primers. This is then a one-step PCR with in total six primers, two adapter sequences, two extension sequences and two amplification sequences. It is sufficient to use the adapter primers and the extension primers at low concentrations and to thus generate an insofar low amount of intermediate product. The intermediate product needs not to be present in a homogeneous form, thus expensive optimizations are not required. Because of the shortness of the amplification primers, even for the amplification to the high amount of nucleic acid end sequences, no optimizations are needed.

Alternatively to the above embodiment and having an independent importance a variant comprising two PCR steps is presented. Therein, the steps a) and b) in a method step A) are performed in a pre-PCR solution containing the nucleic acid base sequence, the adapter primers and the extension primers for a defined first number of cycles, and the step c) in a method step B) is performed in a main PCR solution containing the PCR product from the step A) and the amplification primers for a defined second number of cycles. Step A) may be performed in a reaction volume being ½ to 1/10 of the reaction volume of step B). In step A) will then be generated, due to the lower volume, a higher concentration of intermediate product, or a distinctly lower amount of nucleic acid base sequence can be used. By the dilution by means of PCR starting volume at the transition from step A) to step B), in turn the adapter primers and the extension primers are substantially diluted with the consequence of an increase in the probability of the incorporation of variations and/or modifications in the nucleic acid end sequences via the amplification primers.

In detail, the procedure in the first above alternative may be that the PCR is performed in a reaction volume of 10 to 100 μl preferably 20 to 40 μl with 0.01 to 100 pg, preferably 1 to 50 pg nucleic acid base sequence, 0.05 to 10 μM, preferably 0.1 to 5 μM adapter primer, and 0.005 to 0.5 μM, preferably 0.001 to 0.1 μM extension primer, wherein after a defined number of starting cycles 0.01 to 10 μM, preferably 0.1 to 10 μM amplification primer are added, and wherein by means of a defined number of subsequent cycles the amplified nucleic acid end sequence is produced. In the second above alternative, the following reaction conditions are recommended: step A): reaction volume <10 μl; 0.001 to 5 pg, preferably 0.01 to 1 pg nucleic acid base sequence; 0.05 to 10 μM, preferably 0.1 to 5 μM adapter primer, and 0.05 to 10 μM, preferably 0.1 to 5 μM extension primer; first number of cycles 10 to 30, preferably 15 to 25, step B): reaction volume 10 to 100 μl, preferably 15 to 50 μl, obtained by complementing the solution from step A) with PCR starting solution; 0.01 to 10 μM, preferably 0.1 to 5 μM amplification primer; second number of cycles 15 to 50, preferably 20 to 40.

The invention further teaches the use of the method according to the invention for the production of nucleic acids for the cell-free in vitro protein biosynthesis, in particular in prokaryotic systems, preferably in a translation system of Escherichia coli D10.

A method according to the invention can advantageously be used for the selective amplification of a defined nucleic acid base sequence from a nucleic acid library. This permits a characterization of gene sequences, wherein the gene sequence is used as a nucleic acid base sequence and wherein the obtained protein is analyzed with regard to structure and/or function. The background of this aspect of the invention is that for many genes the sequences are known, not however the structure and function of the protein coded thereby. Thus elements of a gene library of which only the sequence as such is known can be examined for their function in an organism. The examination of the structure and function of the obtained protein follows the conventional working methods of the biochemistry.

By the method according to the invention nucleic acids may be obtained, which contain a nucleic acid base sequence coding for the protein and a ribosomal binding sequence and as an option one or several sequences of the group comprising “promoter sequence, transcription terminator sequence, expression enhancer sequence, stabilization sequence and affinity tag sequence”. An affinity tag sequence codes for a structure having a high affinity for (in most cases immobilized) binding sites in separation systems for the purification. Thus an easy and highly affinitive separation of proteins not containing the affinity tag is possible. An example therefore is Strep-tag II, a peptide structure of 8 amino acid residues with affinity to StrepTactin. A stabilization sequence codes for a structure which either itself or after binding to a specific binding molecule being specific for the structure causes a stabilization against degradation, in particular by nucleases. A stabilization of a nucleic acid (end) sequence may also take place by that on one end, preferably on both ends, a biotin group is incorporated which can be reacted with streptavidin. This incorporation may be effected by using primers carrying biotin, in particular amplification primers. An expression enhancer sequence increases the translation efficiency compared to a nucleic acid without an expression enhancer sequence. For instance (non-translated) spacers may be used for this purpose. A transcription terminator sequence terminates the RNA synthesis. An example is the T7 phage gene 10 transcription terminator. Transcription terminator sequences can also stabilize against degradation by 3′ exonucleases. Advantageous relative arrangements of the above sequence elements with respect to each other can be generalized from the following examples of execution.

In the following, the invention is explained in more detail, based on examples representing preferred embodiments only.

### Methods:

### PCR:

The PCR was performed in a reaction volume quantified in the examples with 10 mM Tris-HCl (pH 8.85 at 20° C.), 25 mM KCl, 5 mM (NH₄)2SO₄, 2 mM MgSO₄, 0.25 of every dNTP, 3 U Pwo DNA polymerase (Roche) and the amount of nucleic acid base sequence specified in the examples. The cycles were performed for 0.5 min at 94° C., 1 min at 55° C. and 1 min at 72° C.

### In Vitro Expression:

In vitro experiments were made according to the document Zubay, G.; Annu. Rev. Genet. 7:267-287 (1973) with the following modifications. The Escherichia coli S-30 lysate was supplemented with 750 U/ml T7 phages RNA polymerase (Stratagene) and 300 μM [14C] Leu (15 dpm/pmol, Amersham). PCR products and control plasmids were used in concentrations of 1 nM to 15 nM. The reactions were performed at 37° C., the course being monitored by that at subsequent times 5 μl aliquots were taken from the reaction mixture and the incorporation of [¹⁴C]Leu was estimated by means of TCA precipitation. Further 10 μl aliquots were taken for the purpose of an analysis of the synthesized protein by means of SDS-PAGE, followed by an autoradiography in a phosphoimager system (Molecular Dynamics).

### Plasmid Construction:

A high copy derivative of the plasmid pET BH-FABP (Specht, B. et al.; J. Biotechnol. 33:259-269 (1994)) coded for bovine heart fatty acid binding protein, called pHMFA, was constructed. A fragment of pET BH-FABP was produced by digestion with the endonucleases Sphl and EcoRI and inserted into the vector pUC18. With regard to the sequences being relevant for the synthesis of H-FABP, the plasmid pHMFA is identical with the original plasmid. It should be noted that the linearized plasmid does not behave in a better way than the circular plasmid.

Construction of Nucleic Acids with Different Sequence Regions Upstream of the Promoter:

The plasmid pHMFA served as a matrix for the construction of nucleic acids with different sequence regions upstream of the promoter. The constructs (see examples) FA1, FA2 and FA4 with 0, 5 and 249 base pairs upstream of the promoter were generated with the primers P1, C1 and P2 as well as with the downstream primer P3. The construct FA3 with a sequence region of 15 base pairs upstream of the promoter was obtained by digestion of FA4 with the endonuclease Bgl II. The control plasmid pHMFA(EcoRV) with a sequence region of 3.040 base pairs was obtained by digestion of the plasmid with EcoRV. All products were purified by agarose gel electrophoresis, followed by gel extraction by means of the “High Pure PCR Product Purification Kit”.

### Affinity Purification:

The purification of the fatty acid binding protein containing Strep-tag II (Voss, S. et al.; Protein Eng. 10:975-982 (1997)) was performed by means of affinity chromatography according to manufacturer's instructions (IBA Goettingen, Germany), with the deviation of a reduced volume of the affinity column (200 μl). The reaction mixture of the coupled transcription/translation was centrifuged for a short time and then applied to the column. Isolated fractions were analyzed by TCA precipitation and audioradiography after SDS-PAGE (see above).

### H-FABP Activity Asssay:

The complete reaction mixture with H-FABP synthesized in vitro was examined for the activity of the binding of oleic acid. Various volumes (0 to 30 μg) were filled up to 30 μl with reaction solution without H-FABP and diluted with translation buffer (50 mM HEPES pH 7.6, 70 mM KOAc, 30 mM NH₄Cl, 10 mM MgCl₂, 0.1 mM EDTA, 0.002% NaN₃ so to obtain an end volume of 120 μl. After addition of 2 μl 5 mM [9,10(n)-³H] oleic acid (Amersham) with a specific activity of 1,000 dpm/pmol, the samples were incubated for one hour at 37° C. 50 μl of the samples were used for removal of unbound oleic acid by means of gel filtration (Micro Bio-Spin Chromatography columns; Bio-Rad). The ³H radioactivity of the eluted fractions was measured by means of a scintillation counter.

### Analysis of the Stability of the Nucleic Acids:

Radioactively marked nucleic acids were synthesized according to the above conditions, however in presence of 0.167 μCi/μl [α-³⁵S] dCTP. The marked nucleic acids were used in a coupled transcription/translation, reaction volume 400 μl. 30 μl aliquots were taken at subsequent times. After addition of 15 μg ribonuclease A (DNAse-free, Roche), they were incubated for 15 min at 37° C. Another incubation for 30 min at 37° C. was performed after addition of 0.5% SDS, 20 mM EDTA and 500 μg/ml proteinase K (Gibco BRL) in a total reaction volume of 60 μl. The remaining PCR products were further purified by means of ethanol precipitation and then subjected to a denaturating electrophoresis (5.3% polyacrylamide, 7 M urea, 0.1% SDS, TBE). The dried gel was passed for the quantification of the radioactivity through a phosphoimager system (Molecular Dynamics).

### Sequences:

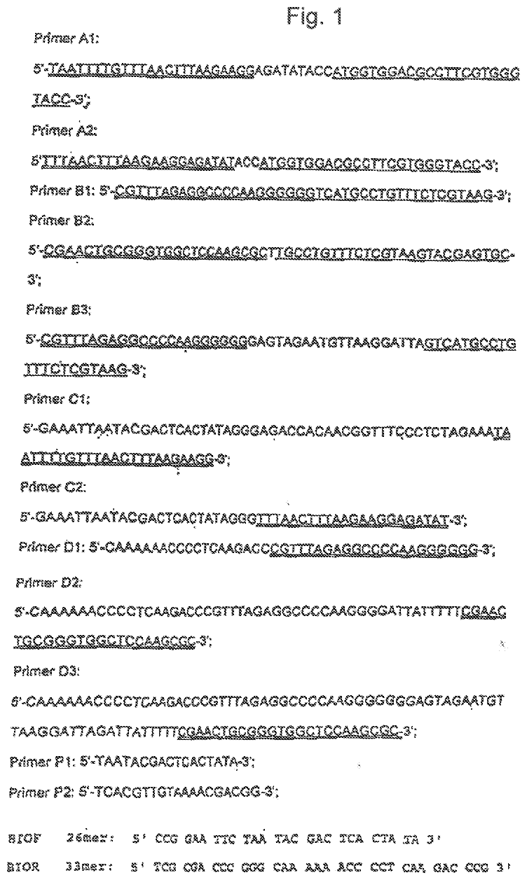

The employed primer sequences are shown in FIG. 1.

### EXAMPLE 1

### PCR with Four Primers

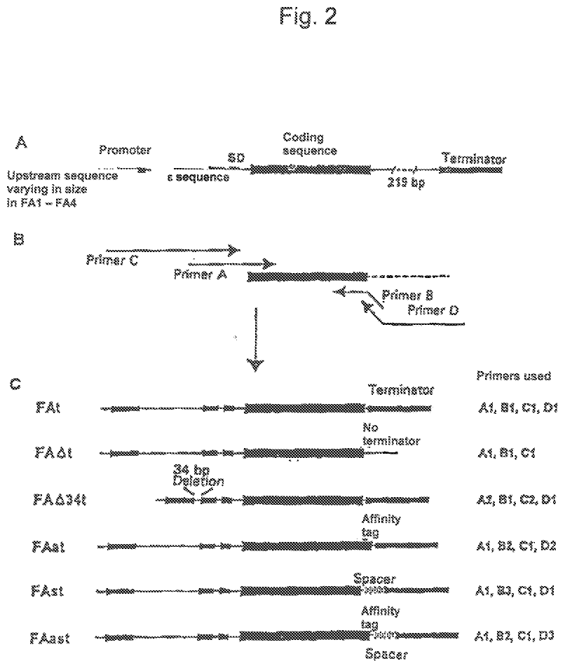

In FIG. 2 is shown a diagrammatic representation of a one-step PCR according to the invention with four primers. In the center, the nucleic acid base sequence coding for a protein can be seen, said nucleic acid base sequence comprising the complete coding sequence for H-FABP (homogeneous and functionally active fatty acid binding protein from bovine heart), obtained as a 548 bp restriction fragment from pHMFA by digestion by means of the endonucleases Ncol and BamHI (and a 150 bp sequence on the 3′ end which is neither translated nor complementary to an adapter primer or extension primer). Thereto the two adapter primers A and B are hybridized, which have ends being homologous with the ends of the nucleic acid base sequence. The adapter primer A further contains a ribosomal binding sequence. To the outside ends of the adapter primers A and B are hybridized the extension primers C and D. The extension primer C comprises the T7 gene 10 leader sequence including the T7 transcription promoter as well as upstream a sequence of for instance 5 nucleotides. The extension primer D comprises the T7 gene 10 terminator sequence.

### EXAMPLE 2

### Efficiency of the H-FABP Synthesis in Dependence from the Sequence Region Upstream of the Promoter

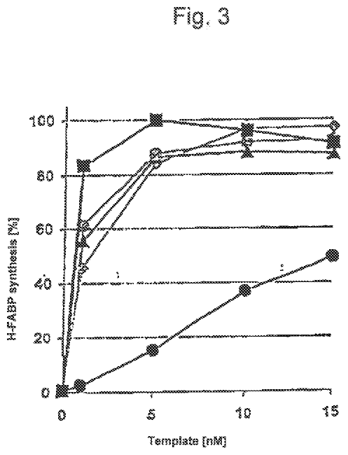

Four PCR products (FA1 to FA4) with different sequence regions upstream of the promoter (0, 5, 15, 250 base pairs) and the linearized control plasmid pHMFA(EcoRV) with 3,040 bp upstream of the promoter were examined in different concentrations (1, 5, 10 and 15 mM) for in vitro transcription/translation. FIG. 3 shows that all sequence regions except 0 (lower curve) cause an increase in the protein synthesis. Five base pairs already are sufficient.

### EXAMPLE 3

### Improvement of the H-FABP Synthesis by the Phage T7 Gene 10 Transcription Terminator/5′ Leader Sequence Phage T7 Gene 10

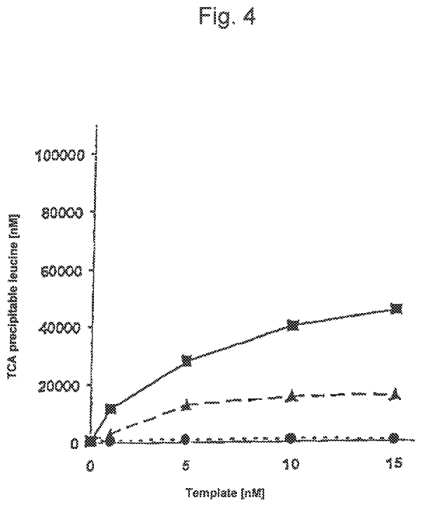

In FIG. 4 can be seen that by the phage T7 gene 10 transcription terminator the synthesis can be at least improved by factor 2.8. The triangles are for FAΔt, and the squares for FAt (see also FIG. 2).

Further, it can be seen from FIG. 4 that by the deletion of 34 bp between the beginning of transcription and the epsilon sequence (Olins, P. O. et al.; Escherichia coli, J. Biol. Chem. 264:16973-16976 (1989)) leads to a suppression of a product generation. The circles are for this variant FAA34 (see also FIG. 2).

### EXAMPLE 4

### Influence of the Position of the Transcription Terminator Sequence

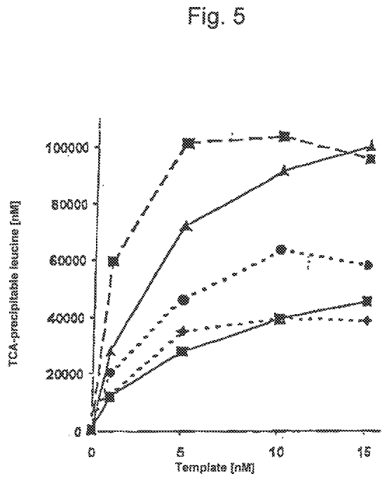

For examining the influence of the position of the terminator sequence, the products FAst and FAast were produced (see FIG. 2). Both are identical with FAt and FAat, with the exception that a 22 bp spacer sequence is introduced between the stopcodon and the terminator by means of different primers. In FIG. 5 can be seen that the spacer sequence causes an approximate 2-fold increase in the expression.

Further, it can also be seen in FIG. 5 from a comparison of FAt and FAat that an affinity tag has nearly no influence on the expression.

### EXAMPLE 5

### PCR from a Complex DNA Mixture

The effectivity and specificity of the method according to the invention was examined in presence of a high amount of competitive DNA. A PCR for FAst was performed according to the above descriptions, with the following exceptions: the nucleic acid base sequence was performed in concentrations from 0.16 to 20 pg/50 μl reactor volume, and the reactions were supplemented with 0.83 μg chromosomal DNA from Escherichia coli, ultrasonically treated for 5 min. It was found that neither the quality nor the quantity of the PCR product was influenced by the presence of a 5 million-fold excess of competitive DNA.

### EXAMPLE 6

### Affinity Purification with Strep-Tag II

A reaction mixture of 10 μg of the radioactively marked FAast was subjected to the affinity purification. Approximately 81% of the applied material were obtained from the column, and 67% could be gained as a pure product in the elution fractions (calculated from TCA precipitation of the fractions of the affinity column).

### EXAMPLE 7

### Activity of the PCR Product

Samples of H-FABP, synthesized either by means of the plasmid or as PCR product FAast were examined together with regard to the binding activity for oleic acid. After the transcription/translation, various volumes of 0 to 330 pmol of non-marked H-FABP were examined in a binding assay according to the above description of methods. The activities were found as being identical, irrespective of the way of production.

### EXAMPLE 8

### Stability of the PCR Product

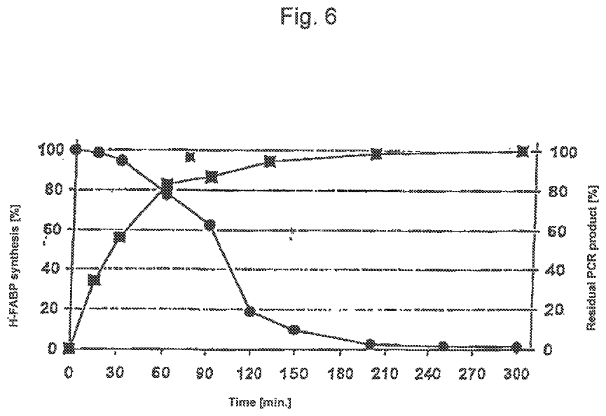

For examining whether the stability of the PCR product would possibly limit the effectivity of the expression, the decrease of the PCR product FAast was measured. For this purpose, the radioactively marked product was used. In certain time intervals, aliquots of the reaction mixture were taken and examined by means of denaturated polyacrylamide gel electrophoresis. The amount of remaining PCR product was quantified by scanning the radioactivity of the gel and compared to the time course of the protein synthesis, measured by scanning the radioactivity of H-FABP in the gel after separation of the reaction mixtures by means of SDS-PHAGE. The results are shown in FIG. 6. It can be seen that the half-life period of the PCR product is approximatel 100 min, which corresponds to the time when the H-FABP synthesis reaches a horizontal line.

### EXAMPLE 9

### Optimized Conditions for a PCR with Four Primers

In Table I are summarized optimized conditions for a PCR with four primers in a reaction volume of 25 μl.

TABLE I

| | Concentration in |
|---|---|
| Reaction component | the reaction |
| a) Reaction components | |
| PCR buffer for PWO polymerase (Roche) | Acc. to supplier |
| Desoxynucleotidetriphosphates dATP, dCTP, dgTP | 0.25 mM |
| and dTTP | |
| Adapter primer a (55 nucleotides) | 0.1 μM |
| Adapter primer b (51 nucleotides) | 0.1 μM |
| Extension primer c (75 nucleotides) | 0.4 μM |
| Extension primer d (95 nucleotides) | 0.4 μM |
| Template: coding sequence for fatty acid binding | 10 pg/25 μl |
| protein/restriction fragment from pHM18FA | |
| (Ncol/BamHI) | |
| PWO DNA polymerase (Rocke | 1.5 U/25 μl |
| b) Temperature program | |
| Temperature cycle | |
| Segment 1 30 sec 94° C. | |
| Segment 2 60 sec 55° C. | |
| Segment 3 60 sec 72° C. | |
| 60 cycle repetitions | |

Example 10: PCR with six primers.

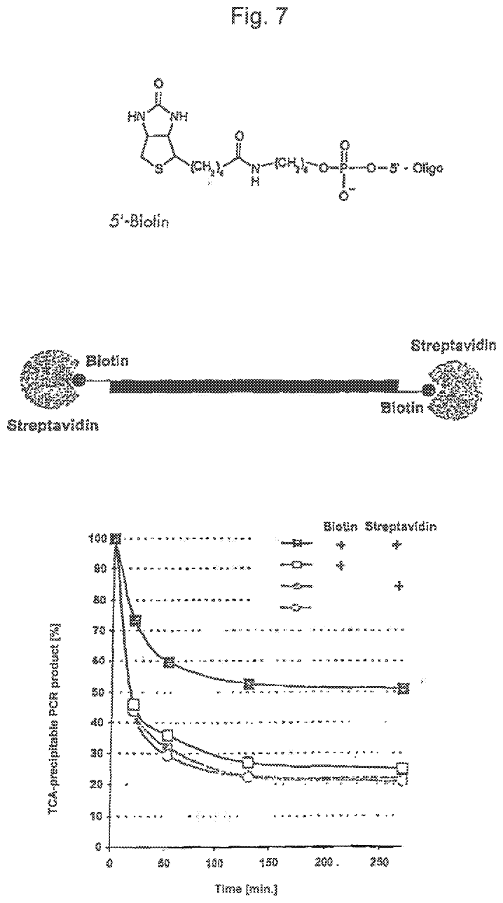

With the materials from example 9, however with two additional amplification primers e (26 nucleotides) and f (33 nucleotides) and an increased adapter primer concentration of 0.2 μM, varying extension primer concentrations were adjusted. With regard to the amplification primers, reference is made to FIG. 1, BIOR and BIOF. BIOF is a biotin-marked forward primer and BIOR a biotin-marked reverse primer. The structure is shown in FIG. 7.

A minimum demand of expensive extension primers resulted, if first 25 cycles without amplification primer and then another 25 cycles with amplification primer were performed. The concentration of the extension primer could be reduced by the use of the amplification primers down to 0.025 μM, a factor of approximately 1/20, with nevertheless improved homogeneity and yield of PCR product.

These advantages are based on that the probability of the generation of intermediate products in high concentrations is strongly reduced with the use of the six primers, since the primers required for the generation of the intermediate products are used in low concentrations. Intermediate products thus cannot be exponentially enriched with the amplification primers.

### EXAMPLE 11

### PCR with Six Primers in Two Steps

In principle, the materials are used as described above. First, a pre-PCR is performed in a reaction volume of 5 μl with 0.1 pg nucleic acid base sequence, with 0.3 μM adapter primer and 0.5 μM extension primer over 20 cycles. Then the reaction solution thus obtained is diluted with PCR starting volume to 25 μl. Then amplification primer is added to an end concentration of 0.5 μM. Finally, it is amplified for another 30 cycles.

### EXAMPLE 12

### Stabilization of a Nucleic Acid with Biotin

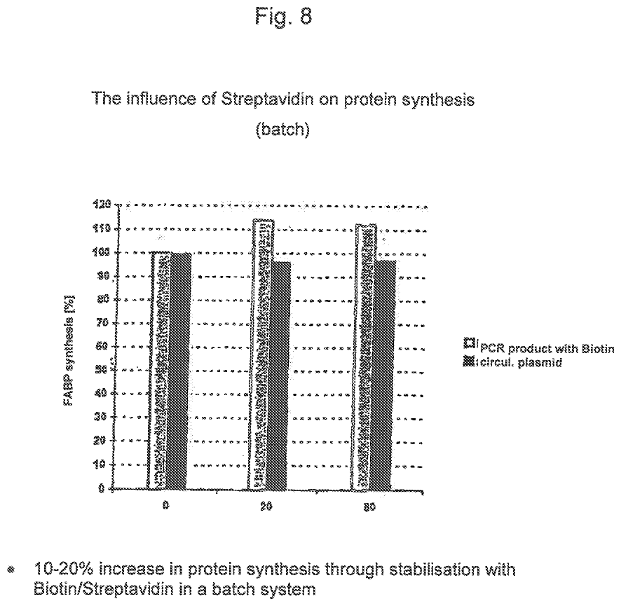

By using the primers BIOF and BIOR in a PCR with 6 primers, as described above, a nucleic acid was produced, and the decomposition thereof as a function of the time and the improvement of the protein synthesis were examined. This is shown in FIGS. 7 and 8. It can be seen that with biotin, in particular after reaction with streptavidin, a substantially better stability is obtained. This also leads to a protein synthesis being higher up to 20%.

Irrespective of the above examples, it has to be noted that with the method according to the invention, variations of the sequences are also very easily possible by mutations, for instance by using Taq polymerase and/or modified reaction conditions. If this is not desired, preferably Pwo or Pfu can be used which function in a more precise manner and have proof-reading activity.

1. A method for preparative production of long nucleic acids by means of PCR and involving the following hybridization steps:
a) a nucleic acid base sequence is hybridized on the 3′ and 5′ ends with an adapter primer,
b) the product from step a) is hybridized on the 3′ and 5′ ends with an extension primer containing an extension sequence,
wherein a nucleic acid sequence enlarged by the extension sequences and amplified on the 3′ and 5′ ends of the nucleic acid base sequence is formed from the nucleic acid base sequence.

2. A method according to claim 1, wherein the product from step b) is hybridized in a step c) on the 3′ and 5′ ends with one amplification primer each, an amplified nucleic acid end sequence being formed.

3. A method according to claim 1 or 2, wherein the adapter primers contain <70 nucleotides, wherein the extension primers contain ≥70 nucleotides, and/or wherein the amplification primers contain <70 nucleotides.

4. A method according to one of claims 1 to 3, wherein the steps a), b) and as an option the step c) are performed in a PCR solution containing the nucleic acid base sequence, the adapter primers, the extension primers and as an option the amplification primers.

5. A method according to one of claims 1 to 4, wherein the steps a) and b) in a method step A) are performed in a pre-PCR solution containing the nucleic acid base sequence, the adapter primers and the extension primers for a defined first number of cycles, and wherein the step c) in a method step B) is performed in a main PCR solution containing the PCR product from the step A) and the amplification primers for a defined second number of cycles.

6. A method according to one of claims 1 to 4, wherein the PCR is performed in a reaction volume of 10 to 100 μl, preferably 20 to 40 μl with 0.01 to 100 pg, preferably 1 to 50 pg nucleic acid base sequence, 0.05 to 10 μM, preferably 0.1 to 5 μM adapter primer, and 0.005 to 0.5 μM, preferably 0.001 to 0.1 μM extension primer, wherein after a defined number of starting cycles 0.01 to 10 μM, preferably 0.1 to 5 μM amplification primer are added, and wherein by means of a defined number of subsequent cycles the amplified nucleic acid end sequence is produced.

7. A method according to claim 5, comprising the following reaction conditions:
step A): reaction volume <10 μl; 0.001 to 5 pg, preferably 0.01 to 1 pg nucleic acid base sequence; 0.05 to 10 μM, preferably 0.1 to 5 μM adapter primer, and 0.05 to 10 μM, preferably 0.1 to 5 μM extension primer; first number of cycles 10 to 30, preferably 15 to 25,
step B): reaction volume 10 to 100 μl, preferably 15 to 50 μl, obtained by complementing the solution from step A) with PCR starting solution; 0.01 to 10 μM, preferably 0.1 to 5 μM amplification primer; second number of cycles 15 to 50, preferably 20 to 40.

8. The use of a method according to one of claims 1 to 7 for the production of nucleic acids for the cell-free in vitro protein biosynthesis, in particular in prokaryotic systems, or for in vitro transcription systems.

9. The use according to claim 8 in a translation system of Escherichia coli D10.

10. The use of a method according to one of claims 1 to 7 for the selective amplification of a defined nucleic acid base sequence from a nucleic acid library.

11. The use of a method according to one of claims 1 to 7 for the characterization of gene sequences, wherein the gene sequence is used as a nucleic acid base sequence and wherein the obtained protein is analyzed with regard to structure and/or function.

12. A nucleic acid for cell-free protein biosynthesis systems which contains a base sequence coding for a protein and a ribosomal binding sequence as well as an option one or several sequences of the group comprising “promoter sequence, transcription terminator sequence, expression enhancer sequence, stabilization sequence and affinity tag sequence”.


SEQUENCE LISTING (14 sequences)

SEQ ID NO: 1 - DNA, 57 bp, Artificial
  Primer
  primer_bind (1)..(57)
taattttgtttaactttaagaaggagatataccatggtggacgccttcgtgggtacc

SEQ ID NO: 2 - DNA, 49 bp, Artificial
  Primer
  primer_bind (1)..(49)
tttaactttaagaaggagatataccatggtggacgccttcgtgggtacc

SEQ ID NO: 3 - DNA, 42 bp, Artificial
  Primer
  primer_bind (1)..(42)
cgtttagaggccccaaggggggtcatgcctgtttctcgtaag

SEQ ID NO: 4 - DNA, 51 bp, Artificial
  Primer
  primer_bind (1)..(51)
cgaactgcgggtggctccaagcgcttgcctgtttctcgtaagtacgagtgc

SEQ ID NO: 5 - DNA, 63 bp, Artificial
  Primer
  primer_bind (1)..(63)
cgtttagaggccccaagggggggagtagaatgttaaggattagtcatgcctgtttctcgtaag

SEQ ID NO: 6 - DNA, 75 bp, Artificial
  Primer
  primer_bind (1)..(75)
gaaattaatacgactcactatagggagaccacaacggtttccctctagaaataattttgtttaactttaagaagg

SEQ ID NO: 7 - DNA, 47 bp, Artificial
  Primer
  primer_bind (1)..(47)
gaaattaatacgactcactatagggtttaactttaagaaggagatat

SEQ ID NO: 8 - DNA, 41 bp, Artificial
  Primer
  primer_bind (1)..(41)
caaaaaacccctcaagacccgtttagaggccccaagggggg

SEQ ID NO: 9 - DNA, 72 bp, Artificial
  Primer
  primer_bind (1)..(72)
caaaaaacccctcaagacccgtttagaggccccaaggggattatttttcgaactgcgggtggctccaagcgc

SEQ ID NO: 10 - DNA, 95 bp, Artificial
  Primer
  primer_bind (1)..(95)
caaaaaacccctcaagacccgtttagaggccccaagggggggagtagaatgttaaggattagattatttttcgaactgcgggtggctccaagcgc

SEQ ID NO: 11 - DNA, 17 bp, Artificial
  Primer
  primer_bind (1)..(17)
taatacgactcactata

SEQ ID NO: 12 - DNA, 19 bp, Artificial
  Primer
  primer_bind (1)..(19)
tcacgttgtaaaacgacgg

SEQ ID NO: 13 - DNA, 26 bp, Artificial
  Primer
  primer_bind (1)..(26)
ccggaattctaatacgactcactata

SEQ ID NO: 14 - DNA, 33 bp, Artificial
  Primer
  primer_bind (1)..(33)
tcgcgacccgggcaaaaaacccctcaagacccg